(19) United States
(12) Patent Application Publication
(10) Pub. No.: US 2007/0118061 A1
(43) Pub. Date: May 24, 2007

(54) Arm positioning device

(76) Inventors: William Calfas, Pacific Palisades, CA (US); Karin A. Calfas, Pacific Palisades, CA (US); Bruce W. Davis, Newbury Park, CA (US); Mary C. Davis, Newbury Park, CA (US)

Correspondence Address: HENRICKS SLAVIN AND HOLMES LLP;SUITE 200, 840 APOLLO STREET, EL SEGUNDO, CA, 90245, US

(21) Appl. No.: 11/282,827

(22) Filed: Nov. 18, 2005

(51) Int. Cl. A61F 5/00

(52) U.S. Cl. 602/35

(57) ABSTRACT

Apparatus and methods for positioning arms or other limbs.

### BACKGROUND OF THE INVENTIONS

1. Field of Inventions

The present inventions relate generally to the transportation of persons on backboards, gurneys, stretchers, wheelchairs, stair chairs and the like.

2. Description of the Related Art

There are many instances where a sick, injured or otherwise less than fully mobile person is transported in a prone or seated position by firefighters, emergency medical technicians (“EMTs”), ambulance personnel, hospital personnel, and others (collectively “transporting personnel”). The person is typically placed on a backboard, gurney, stretcher, wheelchair, stair chair or the like (collectively “patient transport devices”) that is carried or pushed by the transporting personnel. For example, an injured person may be placed on a backboard, carried down a flight of stairs, transferred to a gurney, placed in an ambulance, driven to a hospital, removed from the ambulance, and transported to various locations within the hospital on the gurney.

One issue associated with transporting a person on a patient transport device is the position of the person's arms. The person's arms are typically arranged in a transport position that is comfortable and out of harms way. For example, the arms may be bent slightly at elbows and crossed at the wrists near the waist, or bent significantly at elbows and crossed at the wrists over chest. The exact positioning will, of course, depend on the situation. Unfortunately, arms do not always stay in the desired transport position. The arms of unconscious persons will frequently fall over the side of the patient transport device, as shown in FIG. 1, which exposes the person to injury and increases the difficulty associated with transport. Similarly, a conscious child or a conscious adult who is panicked or otherwise disturbed may reach out over the edge of the patient transport device to, for example, grab onto something out of fear. This also exposes the person to injury and increases the difficulty associated with transport. In addition to exposure to injury and transporting personnel difficulties, intravenous connections and other medical devices may be dislodged when a conscious or unconscious person's arms fall, or are otherwise moved, beyond the sides of the patient transport device.

One proposed solution to the problems associated with arm movement is to position the person's hands palm-to-palm and then tape the arms together by winding tape around the wrists a few times. There are a number of shortcomings associated with this proposed solution. For example, it results in discomfort during transport and pain when the tape is removed. Tape may also tear the skin when it is removed, and some people are allergic to tape.

### SUMMARY OF THE INVENTIONS

An arm positioning device in accordance with one embodiment of a present invention includes a first wrist cuff defining a first wrist opening and a second wrist cuff defining a second wrist opening and secured to the first wrist cuff such that the first and second wrist openings are oriented generally transverse to one another. A method in accordance with one embodiment of a present invention includes the step of securing a first arm to a second arm at the wrists such that the wrists cross one another and first and second arms are generally transverse to one another at the wrists.

Such inventions are advantageous for a variety of reasons. For example, such inventions obviate the above-described issues associated with transporting a person on a patient transport device because they allow the person's arms to be maintained in a desirable transport position. More specifically, the inventions orient the person's arms so that they crossed at the wrists near the waist, or over chest, where they are out of harms way. Securely holding the arms in this manner also prevents an unconscious person's arms from falling over the side of the patient transport device, and makes it very difficult for the person to reach out beyond the perimeter of the patient transport device.

The above described and many other features of the present inventions will become apparent as the inventions become better understood by reference to the following detailed description when considered in conjunction with the accompanying drawings.

### BRIEF DESCRIPTION OF THE DRAWINGS

Detailed descriptions of exemplary embodiments of the inventions will be made with reference to the accompanying drawings.

FIG. 1 is a perspective view showing a person being carried on a backboard in conventional fashion.

FIG. 2 is a perspective view showing a person being carried on a backboard with both arms positioned by one embodiment of a present invention.

FIG. 3 is a perspective view showing a person being carried on a backboard with both arms positioned by one embodiment of a present invention.

FIG. 4 is a perspective view showing a person being carried on a backboard with one arm positioned by one embodiment of a present invention.

FIG. 5 is a perspective view showing a person being carried on a gurney with one arm positioned by one embodiment of a present invention.

FIG. 6 is a partial top view showing a person being carried on a gurney with one arm positioned by one embodiment of a present invention.

FIG. 7 is a perspective view showing one embodiment of a present invention in a closed orientation.

FIG. 8 is a top plan view of one embodiment of a present invention in an open orientation.

FIG. 9 is a bottom plan view of one embodiment of a present invention in an open orientation.

FIG. 10 is an exploded perspective view of one embodiment of a present invention.

FIG. 11 is a plan view of a portion of one embodiment of a present invention.

FIG. 12 is a side view of a portion of one embodiment of a present invention.

FIG. 13 is a side view of a portion of one embodiment of a present invention.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions. The present inventions are also applicable to a wide variety of patient transport devices. Two particularly relevant examples of such devices are backboards and gurneys and, accordingly, the illustrated embodiments of the present inventions are discussed primarily in the context of backboards and gurneys. The present inventions are not, however, limited to backboards and gurneys and may be used in combination with other patient transport devices that currently exist, or are yet to be developed. For example, the present inventions are applicable to stretchers, wheelchairs and stair chairs.

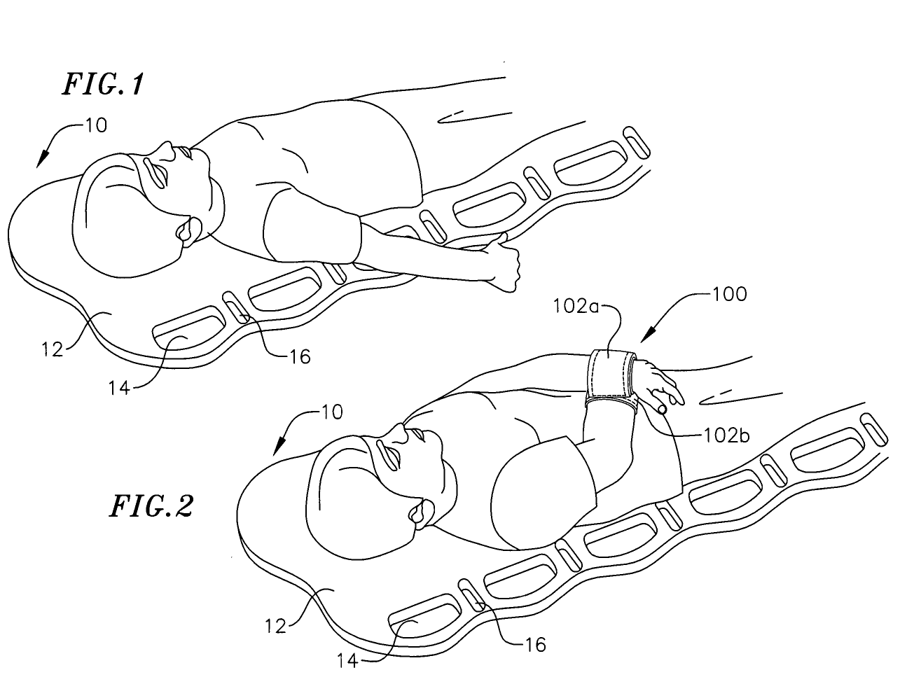

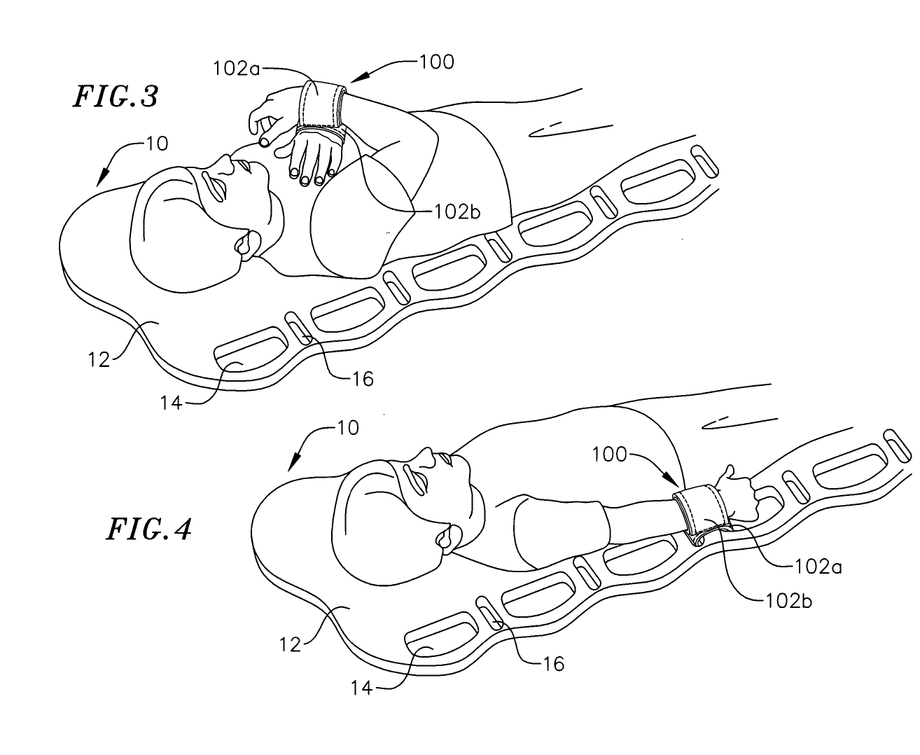

As illustrated in FIGS. 1-3, a person may be transported on a backboard 10 with a support portion 12 and an alternating series of large apertures 14 and small apertures 16. An arm positioning device 100 in accordance with one embodiment of the present invention may be used to quickly, easily and securely orient the person's arms in a transport position that is comfortable and out of harms way, as is shown in FIGS. 2 and 3, in order to prevent the person's arms from moving, either voluntarily or involuntarily, beyond the lateral edges of the backboard 10, as is shown in FIG. 1. To that end, the arm positioning device 100 includes first and second wrist cuffs 102a and 102b that may be used to orient the arms relative to one another in the manner illustrated in FIGS. 2 and 3. The exemplary transport position illustrated in FIG. 2 involves bending the arms slightly at the elbows and crossing the arms at the wrists near the waist. To that end, the arm positioning device 100 is attached to the wrists and secures the arms to one another such that they are crossed at the wrists. Alternatively, in the transport position illustrated in FIG. 3, the exemplary arm positioning device 100 is secured to the wrists and used to orient the arms such that the arms are bent significantly at elbows and crossed at the wrists over chest. The arm positioning device 100 may also be secured to the person in such a manner that the person cannot pull his/her hands through the device or move the device substantially towards an elbow. In the illustrated embodiment, this is accomplished by providing first and second wrist cuffs 102a and 102b that are adjustable in size.

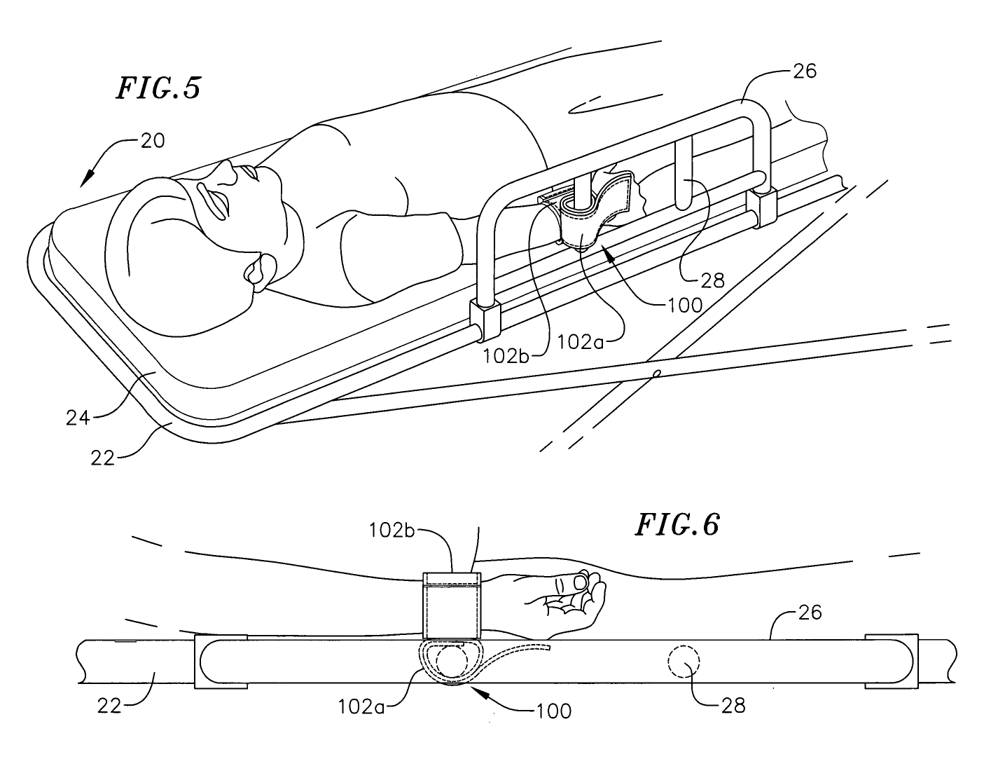

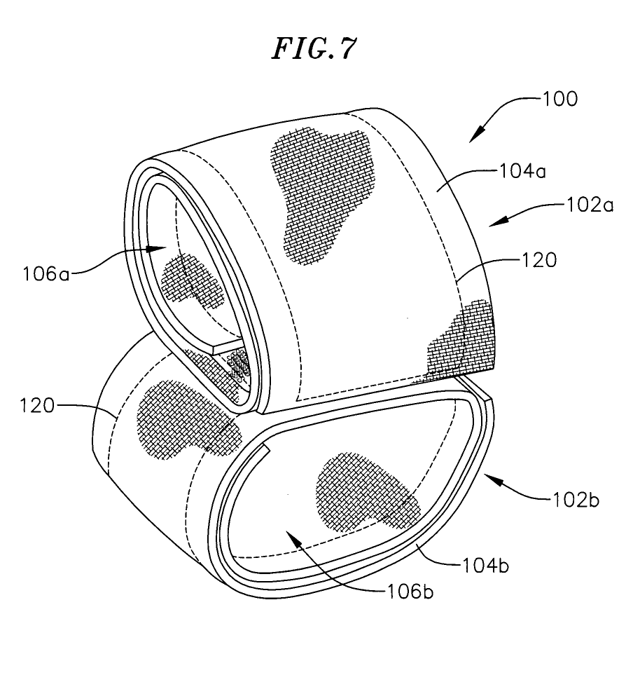

The relative orientation of the first and second wrist cuffs 102a and 102b and the ability to adjust their size also allows the arm positioning device 100 to secure a single arm to a patient transport device in order to hold the arm in a medically necessitated orientation. As illustrated for example in FIG. 4, the arm positioning device 100 may be used to quickly and easily secure a person's wrist to the backboard 10 near the person's hip. More specifically, the wrist cuff 102a is secured to a portion of the backboard between one of the large apertures 14 and an adjacent small aperture 16, while the wrist cuff 102b is secured to the wrist. Securing the wrist to the backboard 10 in this manner is especially useful in those instances where medical personnel intend to make an intravenous connection to the person by way of a vein in the top of the hand. The arm may be rotated 180 degrees if access to a vein in the forearm is desired. The construction of the arm positioning device 100 also allows to wrist to be quickly and easily disconnected when removing the patient from the backboard 10 is desired. Turning to FIGS. 5 and 6, the exemplary arm positioning device 100 may also be used to similarly secure an arm to a gurney 20 that has a frame 22, a pad 24, and a side rail 26 with a plurality of vertically extending posts 28. The wrist cuff 102a may, for example, be secured to one of the vertical posts 28, while the wrist cuff 102b is secured to the wrist. Here too, the arm may be oriented with the palm facing down (FIG. 5) or up (FIG. 6).

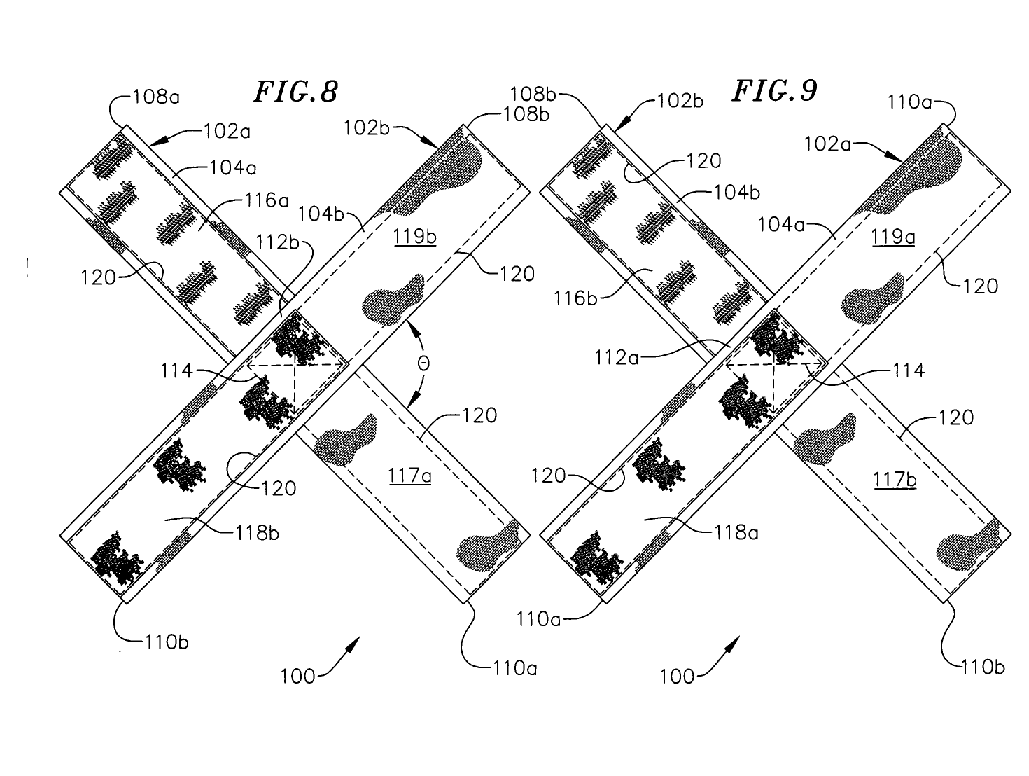

Turning to FIGS. 7-9, the first and second wrist cuffs 102a and 102b of the exemplary arm positioning device 100 include respective straps 104a and 104b, which are secured to one another, and variable size closure mechanisms that allow the wrist openings 106a and 106b defined by the straps to be varied in size when the arm positioning device is placed in the closed orientation (FIG. 7). The straps 104a and 104b, which have free (or “longitudinal”) ends 108a/110a and 108b/110b and mid-portions 112a and 112b where the straps cross one another, are preferably formed from a relatively soft, flexible material (e.g. neoprene, fabric, soft leather, medical foam, or webbing) that will be comfortable for the wearer. Also, although the present inventions are not so limited, the first and second wrist cuffs 102a and 102b of the exemplary arm positioning device 100 are oriented perpendicular to one another. In other words, the angle θ between the straps 104a and 104b (FIG. 8) is 90 degrees and the longitudinal axes of the wrist openings 106a and 106b (FIG. 7) will be perpendicular to one another. The angle θ between the straps 104a and 104b may vary by, for example, up to 45 degrees in some generally transverse (as apposed to perpendicular) implementations.

The straps 104a and 104b may be secured to one another with an attachment device such as stitching 114 (FIGS. 8 and 9). The stitching 114 may be in the illustrated “x within a square” pattern or any other suitable pattern. Other exemplary attachment devices for joining the straps 104a and 104b include, but are not limited to, rivets and adhesive. So connected, the strap mid-portions 112a and 112b are coextensive with one another, and the other portions of the straps 104a and 104b are not.

Although the present inventions are not limited to any particular closure mechanisms, the closures mechanisms in the illustrated embodiment are hook and loop fasteners, such as those commonly sold under the trade name Velcro. Referring more specifically to FIGS. 8 and 9, the straps 104a and 104b include respective strips of hook material 116a and 116b on strap sides 117a and 117b and respective strips of loop material 118a and 118b on the other sides, which are strap sides 119a and 119b. The strips of hook material 116a and 116b and loop material 118a and 118b are each secured to the associated straps with stitching 120 that preferably extends completely around the perimeter of the strip of hook (or loop) material. Other exemplary variable size closure mechanisms include, for example, buckles and ties.

In the illustrated embodiment, the strips of hook material 116a and 116b and loop material 118a and 118b extend from the associated strap free end 108a, 110a, 108b or 110b to the associated strap mid-portions 112a or 112b. The stitching 114 (or other attachment device) extends though both of the straps 104a and 104b and all four of the strips of hook material and loop material 116a, 116b, 118a and 118b. This arrangement creates a considerably stronger connection between the first and second wrist cuffs 102a and 102b than would be the case if the strips of hook material and loop material 116a, 116b, 118a and 118b did not extend into the strap mid-portions 112a or 112b and the stitching 114 only extended through the straps 104a and 104b. The stronger connection prevents the straps 104a and 104b from being separated, or twisted relative to one another, by a combative or otherwise agitated person. As such, the present arrangement facilitates the use of softer, more comfortable strap material (e.g. 4 mil thick neoprene) than would be practicable without it.

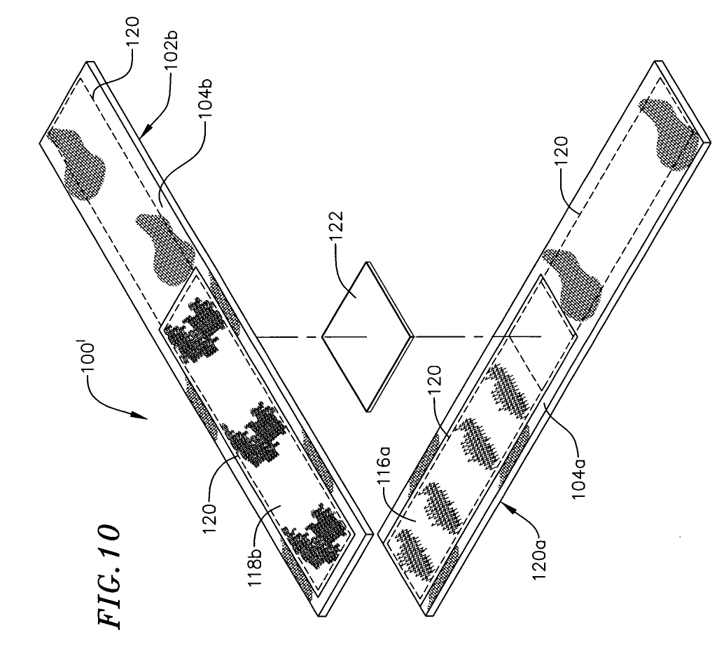

Turning to FIG. 10, the strength of the connection between the straps 104a and 104b is further augmented in the exemplary arm positioning device 100′, which is otherwise identical to arm positioning device 100, through the use of a center piece 122 that is positioned between the straps and is also secured with the stitching 114 or other attachment device. Suitable materials for the center piece 122 include leather and seatbelt-type webbing material.

With respect to adjustability, and as discussed above, the strips of hook material 116a and 116b and loop material 118a and 118b on each of the first and second wrist cuffs 102a and 102b combine to cover, albeit on opposite sides and with overlap at mid-portions 112a and 112b, the entire length of the straps 104a and 104b. As such, relatively large wrist openings 106a and 106b may be formed by bending the straps 104a and 104b from the open orientation illustrated in FIGS. 8 and 9 to a closed orientation where only relatively small portions of the hook material 116a and 116b near the strap ends 108a and 108b are connected to relatively small portions of the loop material 118a and 118b near the strap ends 110a and 110b. Alternatively, relatively small wrist openings 106a and 106b may be formed by bending the straps 104a and 104b from the open orientation illustrated in FIGS. 8 and 9 to a closed orientation where the portions of the hook material 116a and 116b near the strap ends 108a and 108b are connected to portions of the loop material 118a and 118b near the strap mid-portions 112a or 112b (note FIG. 7). Still smaller wrist openings 106a and 106b may be formed by rolling up portions of the straps 104a and 104b with the hook material 116a and 116b and then connecting a region of the hook material spaced inwardly from the strap ends 108a and 108b to the loop material 118a and 118b near the strap mid-portions 112a or 112b (note FIGS. 5 and 6).

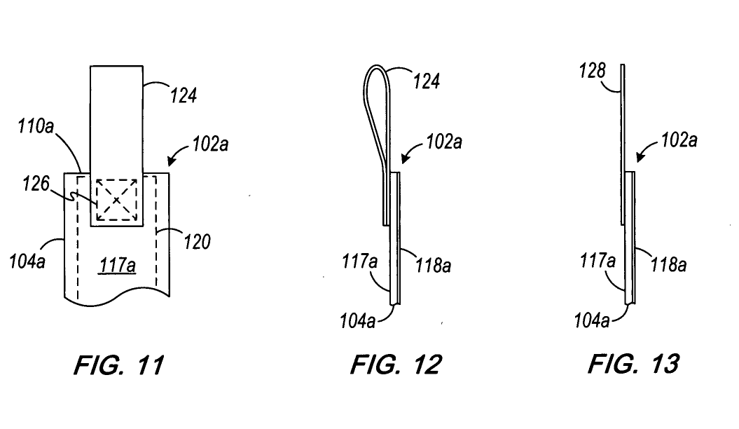

The present inventions may also be provided with structures that allow the transporting personnel to quickly disengage the closure mechanisms and remove the straps from the patient's wrists. As illustrated for example in FIGS. 11 and 12, each of the first and second wrist cuffs 102a and 102b (only wrist cuff 102a is shown) may be provided with a tab 124. The tabs 124 allow the transporting personnel to get a good grip on the longitudinal end 110a of the strap 104a, as well as the longitudinal end 110b of the strap 104b, so that the strips of hook and loop material can be pulled apart. The tabs 124, which are secured to strap sides 117a and 117b with stitching 126, may be of any suitable configuration and material. The exemplary tabs 124 are each in the form of a loop, wherein a length of material is bent back over itself. Both free ends of the length of material are secured to the associated strap with the stitching 126. The loop gives the transporting personnel the option of, for example, squeezing the tab 124 between the thumb and forefinger or running a finger through the opening in the loop. Alternatively, as illustrated in FIG. 13, the exemplary tab 128 is in the form of a strip, wherein one free end of a length of material is secured to the strap sides 117a (and 117b) with stitching 126. Webbing is one example of a suitable material for the tabs 124 and 128.

Although the inventions disclosed herein have been described in terms of the preferred embodiments above, numerous modifications and/or additions to the above-described preferred embodiments would be readily apparent to one skilled in the art. By way of example, but not limitation, the inventions include any combination of the elements from the various species and embodiments disclosed in the specification that are not already described. The straps 104a and 104b, which are each unitary structures, may be formed from multiple pieces that are secured to one another. With respect to usage, the arm positioning devices described above may also be used to secure a leg to a patient transport device at the ankle (in a manner similar to the arm illustrated in FIGS. 4-6) if, for example, the person being transported is having a seizure. Multiple arm positioning devices may be used to secure each of a person's arms in the manner illustrated in FIGS. 4-6 and/or each of a person's legs in the manner discussed above. A combative person might be restrained by securing one arm to a patient transport device the near the person's head and securing the other arm near to the patient transport device near the thigh. A combative person's legs might also be secured to the patient transport device. Here, a total of four of the arm positioning devices would be used. It is intended that the scope of the present inventions extend to all such modifications and/or additions and that the scope of the present inventions is limited solely by the claims set forth below.

We claim:

1. An arm positioning device, comprising:
a first wrist cuff defining a first wrist opening; and
a second wrist cuff defining a second wrist opening and secured to the first wrist cuff such the first and second wrist openings are oriented generally transverse to one another.

2. An arm positioning device as claimed in claim 1, wherein the first and second wrist openings are perpendicular to one another.

3. An arm positioning device as claimed in claim 1, wherein a portion of the first wrist cuff is coextensive with a portion of the second wrist cuff.

4. An arm positioning device as claimed in claim 1, wherein the first and second wrist cuffs are configured such that the size of the first and second wrist openings may be varied.

5. An arm positioning device as claimed in claim 1, wherein
the first wrist cuff comprises a first strap and a first variable size closure mechanism; and
the second wrist cuff comprises a second strap and a second variable size closure mechanism.

6. An arm positioning device as claimed in claim 5, wherein
the first variable size closure mechanism comprises a strip of hook fastener material and a strip of loop fastener material; and
the second variable size closure mechanism comprises a strip of hook fastener material and a strip of loop fastener material.

7. An arm positioning device as claimed in claim 6, wherein
the first strap defines first and second sides, the strip of hook fastener material is on the first side, and the strip of loop fastener material is on the second side; and
the second strap defines first and second sides, the strip of hook fastener material is on the first side, and the strip of loop fastener material is on the second side.

8. An arm positioning device as claimed in claim 6, wherein
the first strap defines a mid-portion and the strips of hook fastener material and loop fastener material extend into the mid-portion; and
the second strap defines a mid-portion and the strips of hook fastener material and loop fastener material extend into the mid-portion.

9. An arm positioning device as claimed in claim 8, wherein
the first and second wrist cuffs are secured to one another by an attachment device that extends though the first strap, the strips of hook fastener material and loop fastener material on the first strap, the second strap, and the strips of hook fastener material and loop fastener material on second strap.

10. An arm positioning device as claimed in claim 9, wherein the attachment device comprises stitching.

11. An arm positioning device as claimed in claim 9, wherein the first and second straps are formed from a relatively soft material.

12. An arm positioning device as claimed in claim 8, wherein
the first strap defines first and second longitudinal end portions and first and second sides, the strip of hook fastener material is on the first side and extends from the first longitudinal end portion to the mid-portion, and the strip of loop material is on the second side and extends from the second longitudinal end portion to the mid-portion; and
the second strap defines first and second longitudinal end portions and first and second sides, the strip of hook fastener material is on the first side and extends from the first longitudinal end portion to the mid-portion, and the strip of loop material is on the second side and extends from the second longitudinal end portion to the mid-portion.

13. An arm positioning device as claimed in claim 5, wherein the first and second straps define longitudinal ends, the arm positioning device further comprising
a first tab secured to the first strap adjacent to one of the longitudinal ends; and
a second tab secured to the second strap adjacent to one of the longitudinal ends.

14. A method of transporting a person having first and second arms on a patient transport device, comprising the step of:
securing the first arm to the second arm at the wrists such that the wrists cross one another and first and second arms are generally transverse to one another at the wrists.

15. A method as claimed in claim 14, wherein
securing the first arm to the second arm comprises securing the first arm to the second arm at the wrists such that the wrists cross one another and the first and second arms are substantially perpendicular to one another at the wrists.

16. A method as claimed in claim 14, wherein
securing the first arm to the second arm comprises securing the first arm to the second arm with an arm positioning device, having first and second wrist cuffs, such that the wrists cross one another and the first and second arms are generally transverse to one another at the wrists.

17. A method as claimed in claim 14, further comprising the step of:
positioning the wrists adjacent to the person's waist.

18. A method as claimed in claim 14, further comprising the step of:
positioning the wrists adjacent to the person's chest.

19. A method of securing a limb to a patient transport device, comprising the steps of:
securing an arm positioning device, having first and second wrist cuffs that are fixedly secured to one another such that the first and second wrist cuffs are generally transverse to one another, to the limb and to the patient transport device.

20. A method as claimed in claim 19, wherein
the limb comprises an arm; and
securing an arm positioning device comprises securing an arm position device, having first and second wrist cuffs that are fixedly secured to one another such that the first and second wrist cuffs are generally transverse to one another, to the arm and to the patient transport device.
